(19) United States
(12) Patent Application Publication
(10) Pub. No.: US 2025/0169822 A1
(43) Pub. Date: May 29, 2025

(54) MULTI-CATHETER SYSTEMS FOR TREATING HEART FAILURE

(71) Applicant: Pulnovo Medical Inc, Las Vegas, NV (US)

(72) Inventors: Wen GU, Las Vegas, NV (US); Cynthia CHEN, Seattle, WA (US)

(21) Appl. No.: 18/970,393

(22) Filed: Dec. 5, 2024

Related U.S. Application Data
(63) Continuation of application No. PCT/US23/68086, filed on Jun. 7, 2023.
(60) Provisional application No. 63/349,975, filed on Jun. 7, 2022.
(60) Provisional application No. 63/397,289, filed on Aug. 11, 2022.
(60) Provisional application No. 63/469,278, filed on May 26, 2023.

(51) Int. Cl. A61B 17/12

(52) U.S. Cl. CPC A61B 17/12045; A61B 17/12109; A61B 17/12136

(57) ABSTRACT

The present disclosure provides a balloon catheter system for treating heart failure in a subject. The balloon catheter systems of the present disclosure form low pressure areas below the subclavian vein and above the renal vein which promotes the return of blood and lymph back to the heart. Additionally, the balloon catheter systems stimulate the vagus nerve, thereby reducing activation of the sympathetic nervous system and improving cardiac remodeling.

### CROSS-REFERENCE

This application is a continuation of International Patent Application No. PCT/US23/68086, filed Jun. 7, 2023, which claims the benefit of U.S. Provisional Patent Application Nos. 63/349,975, filed Jun. 7, 2022, 63/397,289, filed Aug. 11, 2022, and 63/469,278, filed May 26, 2023, each which is incorporated herein by reference in its entirety.

### BACKGROUND

Heart failure is an epidemic disease which affects about 1% to 2% of the population worldwide. Both the etiology and phenotype of heart failure differ largely. Following a cardiac injury (e.g., myocardial infarction, increased preload or afterload), cellular, structural, and neurohumoral modulations occur that affect the phenotype presented. These processes influence cell functions, including intracellular as well as intercellular behavior. Consequently, activation of the sympathoadrenergic and renin-angiotensin-aldosterone systems takes place, leading to adaptive mechanisms, which are accompanied by volume overload, tachycardia, dyspnea, and further deterioration of the cellular functions, which is referred to in the field as the vicious circle of heart failure. There currently exists no heart failure specific clinical signs, and clinical symptoms show progressive deterioration acutely or chronically. As a measure of cellular dysfunction, the level of neurohormones (e.g., norepinephrine) and natriuretic peptides (e.g., NT-pro BNP) increase. For the diagnosis of heart failure, noninvasive (e.g., echocardiography, NMR, NT-pro BNP) and invasive (e.g., heart catheterization, biopsy) diagnostic procedures are implemented. Modulation of the activated systems by ß-blocker, ACE-inhibitors, and/or ARNI can improve outcomes and symptoms in heart failure patients with left ventricular dysfunction. Interventional and surgical therapy options may be performed as well. However, there remains a need for improved systems, devices, and methods of treating heart failure.

### SUMMARY

The present disclosure provides balloon catheter systems for the treatment of heart failure, such as acute decompensated heart failure (ADHF) (i.e., the rapid onset of fluid volume overload).

In an aspect, the present disclosure provides a catheter system for the treatment of a cardiovascular disease. The catheter system may include a first catheter. The first catheter may include a first balloon. The first catheter may include a first lumen configured to inflate the first balloon. The first catheter may include a second lumen. The first catheter may include a third lumen.

The catheter system may include a second catheter. The second catheter may include a second balloon. The second catheter may include an inflation lumen configured to inflate the second balloon. The first catheter may be configured to accept the second catheter for advancement and retraction therethrough. The third lumen of the first catheter may be configured to accept a third catheter for advancement and retraction therethrough. The third catheter may include a third balloon. The third catheter may include an inflation lumen configured to inflate the third balloon.

In some embodiments, the first balloon is configured to be positioned and inflated at or near a renal vein of the subject. In some embodiments, the second balloon is configured to be positioned at or near a subclavian vein of the subject. In some embodiments, the third balloon is a floating balloon. In some embodiments, the third balloon is configured to be positioned and inflated at or near a pulmonary artery of the subject.

In some embodiments, inflation of the second balloon creates a low-pressure area at or near the left internal jugular vein and/or thoracic duct when positioned and inflated therein. In some embodiments, inflation of the second balloon stimulates a vagus nerve of the subject. In some embodiments, inflation of the first balloon creates a low-pressure area at or near a renal vein of the subject. In some embodiments, inflation of the first balloon stimulates a vagus nerve of the subject. In some embodiments, the third balloon monitors at least one hemodynamic parameter of the subject. In some embodiments, the at least one hemodynamic parameter is one or more of heart rate, blood pressure, stroke volume, cardiac output, or total peripheral resistance.

In some embodiments, the first catheter has a distal end port coupled to the second lumen and the second catheter is advanced out of the first catheter through the distal end port. In some embodiments, the first catheter has a side port proximal to a distal end of the first catheter and the third catheter is advanced out of the first catheter through the side port.

In some embodiments, the first balloon has a diameter of about 18 mm to about 25 mm when inflated. In some embodiments, the first balloon has a length about 10 mm when inflated. In some embodiments, the first balloon is configured to be inflated to about 1 atm to about 2 atm of pressure. In some embodiments, the pressure of the first balloon is proportional or relative to the low-pressure at the low-pressure area at or near a renal vein of the subject.

In some embodiments, the second balloon has a diameter of about 8 mm to about 10 mm when inflated. In some embodiments, the second balloon has a length of about 10 mm when inflated. In some embodiments, the second balloon may be inflated to about 1 atm to about 2 atm of pressure.

In some embodiments, the diameter of the first lumen is about 2 Fr. In some embodiments, the diameter of the second lumen is about 7 Fr. In some embodiments, the diameter of the third lumen is about 7 Fr.

In some embodiments, the outer diameter of the second catheter is about 6 Fr. In some embodiments, the length of the second catheter is about 80 cm.

In some embodiments, the second catheter is configured to be connect to a subclavian pressure line. In some embodiments, the subclavian pressure line is configured to measure subclavian vein pressure of the subject. In some embodiments, the subclavian pressure is used to determine whether to inflate or deflate the second balloon.

In some embodiments, the second catheter further comprises a pressure measurement lumen separate from the inflation lumen for the second balloon.

In some embodiments, the outer diameter of the third catheter is about 6 F.

In some embodiments, the third catheter is a pulmonary artery catheter. In some embodiments, the third catheter is configured to connect to one or more of a femoral pressure line or a pulmonary artery pressure line. In some embodiments, the femoral pressure line is configured to measure pressure at a femoral vein. In some embodiments, the pulmonary artery pressure line is configured to measure pressure at the pulmonary artery. In some embodiments, one or more of the measured femoral vein pressure or pulmonary artery pressure is used to determine whether to inflate or deflate the first balloon.

In some embodiments, the third catheter comprises a balloon inflation lumen, a first pressure measurement lumen, and a second pressure measurement lumen. The first pressure measurement lumen may be in fluid communication with a distal end port of the third catheter to facilitate measurement of pressure at the pulmonary artery. The second pressure measurement lumen may be in fluid communication with a side port of the third catheter to facilitate measurement of pressure at the right atria.

In some embodiments, the system comprises the third catheter.

In some embodiments, the catheter system may further include a detachable sheath. In some embodiments, the detachable sheath has a shaft length of about 35 cm. In some embodiments, the detachable sheath is configured to be inserted into a femoral vein of a subject. In some embodiments, the detachable sheath comprises a tapered dilator. In some embodiments, the taper dilator is configured to enlarge a puncture site.

In some embodiments, the catheter system may further include at least one pump configured to couple to one or more of the first, second, or third catheters to inflate and deflate one or more of the first, second, or third balloons.

In some embodiments, the second lumen of the first catheter has a rounded or circular cross-section to accept the second catheter for advancement and retraction therethrough. In some embodiments, wherein the third lumen of the first catheter has a rounded or circular cross-section to accept the third catheter for advancement and retraction therethrough. In some embodiments, the first catheter further comprises a fourth lumen for measuring pressure. The fourth lumen may be in fluid communication with a side port of the first catheter to facilitate measurement of pressure at one or more of the inferior vena cava or a femoral vein.

In some embodiments, one or more of the first catheter, second catheter, or third catheter comprises at least one radiopaque marker.

In some embodiments, one or more of the first catheter, the second catheter, or the third catheter is configured to be advanced through vasculature of the subject over one or more guidewires. In some embodiments, the radiopaque marker may be used to guide one or more of the first catheter, second catheter, or third catheter through the vasculature of the subject to a target location. In some embodiments, the target location is one or more of a renal vein, pulmonary artery, and/or a subclavian vein.

In some embodiments, the cardiovascular disease to be treated is heart failure or acute heart failure.

In another aspect, the present disclosure provides a method for treating a cardiovascular disease using the catheter system of the present disclosure.

The method may include the step of inserting the catheter system into a femoral vein of the subject. The method may include the step of positioning the tip of the first catheter of the catheter system at or near a renal vein of the subject. The method may include the step of positioning the second balloon of the second catheter of the multi-catheter system at or near a subclavian vein of the subject. The method may include the step of inflating the first balloon and the second balloon, wherein inflation of the second balloon creates a low-pressure area at the left internal jugular vein and thoracic duct of the subject, and wherein inflation of the first balloon creates a low-pressure area at the renal vein of the subject.

In some embodiments, the method may further include the step of measuring the pressure at the subclavian vein of the subject using the catheter system. In some embodiments, the method may further include the step of adjusting the inflation of one or more of the first balloon or the second balloon in response to the measured subclavian vein pressure. In some embodiments, adjusting the inflation of the first balloon modulates the pressure at the renal vein. In some embodiments, adjusting the inflation of the second balloon modulates the pressure at the left internal jugular vein and thoracic duct. In some embodiments, inflation of the second balloon stimulates a vagus nerve of the subject. In some embodiments, inflation of the first balloon stimulates a vagus nerve of the subject.

In some embodiments, inflating the first balloon includes maintaining inflation of the first balloon for 20-40 seconds before deflating the first balloon. In some embodiments, inflating the second balloon includes maintaining inflation of the second balloon for 10-30 seconds before deflating the second balloon.

In some embodiments, positioning the tip of the first catheter at or near the renal vein comprises advancing the first catheter through vasculature of the subject over a guidewire. In some embodiments, positioning the second balloon at or near the subclavian vein of the subject comprises advancing the second catheter through vasculature of the subject over a guidewire.

In some embodiments, the method may further include the step of inserting a third catheter comprising a third balloon into the third lumen of the catheter system. In some embodiments, the method may further include the step of positioning the third balloon of the third catheter at or near the pulmonary artery. In some embodiments, the method may further include the step of inflating the third balloon. In some embodiments, the method may further include the step of measuring a pulmonary artery pressure of the subject.

In some embodiments, positioning the third balloon of the third catheter at or near the pulmonary artery comprises advancing the third catheter through vasculature of the subject over a guidewire.

In some embodiments, the method further comprises monitoring at least one hemodynamic parameter of the subject with the third catheter. The at least one hemodynamic parameter may be one or more of heart rate, blood pressure, stroke volume, cardiac output, or total peripheral resistance.

In some embodiments, the method further comprises monitoring pressure at a right atria of the subject with the catheter system. In some embodiments, the method further comprises monitoring pressure at one or more of an inferior vena cava or femoral vein with the catheter system.

In some embodiments, the cardiovascular disease to be treated is heart failure or acute heart failure.

In another aspect, the present disclosure provides a method for treating a cardiovascular disease of a subject. The method may include the step of advancing a first catheter into a femoral vein of the subject. The method may include the step of positioning a first balloon of the first catheter at an inferior vena cava of at or near a renal vein of the subject. The method may include the step of advancing a second catheter through the first catheter. The method may include the step of positioning a second balloon of the second catheter at a subclavian vein of the subject. The method may include the step of inflating the second balloon, thereby creating a low-pressure area at one or more of the left internal jugular vein or the thoracic duct of the subject. One or more of the first catheter or the second catheter may be advanced through vasculature of the subject over one or more guidewires

In some embodiments, inflating the second balloon includes stimulating a vagus nerve of the subject or minimizes sympathetic nerve activation of the subject.

In some embodiments, the method may further include the step of inflating the first balloon at or near the renal vein of the subject. In some embodiments, inflating the first balloon creates a low-pressure area at the renal vein. In some embodiments, inflating the first balloon includes stimulating a vagus nerve of the subject.

In some embodiments, inflating the first balloon includes maintaining inflation of the first balloon for about 20 seconds to about 40 seconds before deflating the first balloon. In some embodiments, inflating the second balloon includes maintaining inflation of the second balloon for about 10 seconds to about 30 seconds before deflating the second balloon.

In some embodiments, the method further comprises advancing a third catheter comprising a third balloon at or near the pulmonary artery, inflating the third balloon, and monitoring a pulmonary artery pressure of the subject. In some embodiments, the method further comprises monitoring at least one hemodynamic parameter of the subject with the third catheter. The at least one hemodynamic parameter may be one or more of heart rate, blood pressure, stroke volume, cardiac output, or total peripheral resistance. In some embodiments, the method may further comprise measuring pressure at a pulmonary artery or a right atria. In some embodiments, the method further comprises visualizing the third catheter with aid of at least one radiopaque marker of the third catheter.

In some embodiments, the method further comprises measuring pressure at the subclavian vein of the subject and adjusting inflation of one or more of the first balloon or second balloon in response to the measured subclavian vein pressure. Adjusting the inflation of the first balloon may modulate the pressure at the renal vein. Adjusting the inflation of the second balloon may modulate the pressure at the left internal jugular vein and thoracic duct.

In some embodiments, the method may further comprise measuring pressure at one or more of the inferior vena cava or a femoral artery.

In some embodiments, the method further comprises visualizing one or more of the first catheter or second catheter with aid of at least one radiopaque marker of the first catheter or the second catheter

In some embodiments, the cardiovascular disease to be treated is heart failure or acute heart failure.

In another aspect, the present disclosure provides a method for treating a cardiovascular disease of a subject. The method may comprise creating a low-pressure area at a left internal jugular vein and/or thoracic duct of the subject and creating a low-pressure area at a renal vein of the subject.

In some embodiments, creating the low-pressure area at the renal vein of the subject comprises intermittently occluding an inferior vena cava of the subject at or near the renal vein. Intermittently occluding the inferior vena cava of the subject at or near the renal vein may comprise advancing a first catheter from a femoral vein of the subject and to at or near a renal vein and inflating a balloon of the first catheter at an inferior vena cava. Inflating the balloon of the first catheter may comprise maintaining inflation of the balloon for about 20 seconds to about 40 seconds before deflating said balloon.

In some embodiments, creating the low-pressure area at the left internal jugular vein and thoracic duct comprises intermittently occluding a subclavian vein of the subject. Intermittently occluding the subclavian vein may comprise advancing a second catheter from a femoral vein of the subject and to subclavian vein and inflating a balloon of the second catheter at an inferior vena cava. Inflating the balloon of the second catheter may comprise maintaining inflation of the balloon for about 10 seconds to about 30 seconds before deflating said balloon.

In some embodiments, creating the low-pressure area at a left internal jugular vein and/or thoracic duct increases lymphatic reflow of the subject, reduces fluid retention in the body of the subject, or both.

In some embodiments, creating a low-pressure area at a renal vein of the subject promotes one or more of renal circulation, speeds up urination of the subject, or reduced fluid retention in the body of the subject.

In some embodiments, creating the low-pressure area at the left internal jugular vein and/or thoracic duct, creating the low-pressure area at the renal vein duct, or both stimulates a vagus nerve of the subject. Stimulating the vagus nerve of the subject may cause one or more of a subclavian vein, a left internal jugular vein and/or thoracic duct, an inferior vena cava, or a renal vein to expand. Stimulating the vagus nerve of the subject may reduce venous blood backflow to the heart of the subject, reduce heart pumping burden of the subject, or both.

In some embodiments, the cardiovascular disease to be treated is heart failure or acute heart failure.

In another aspect, the present disclosure provides a method for treating a cardiovascular disease of a subject. The method may comprise intermittently occluding one or more veins to reduce venous blood backflow to the heart of the subject and reduce heart pumping burden. Intermittently occluding the one or more veins may comprise one or more of: intermittently occluding a subclavian vein of the subject, thereby creating a low-pressure area at a left internal jugular vein and/or thoracic duct of the subject; or intermittently occluding an inferior vena cava of the subject, thereby creating a low-pressure area at a renal vein of the subject. The subclavian vein may be occluded for a period of 10 to 30 seconds. The inferior vena cava may be occluded for a period of 20 to 40 seconds.]

The one or more veins may be intermittently occluded by expanding one or more balloons of a catheter system at or near the one or more veins. Intermittently occluding the one or more veins may stimulate a vagus nerve of the subject. Intermittently occluding the one or more veins may increase lymphatic reflow of the subject, reduce fluid retention in the body of the subject, promote renal circulation, or speed up urination of the subject. In some embodiments, the cardiovascular disease to be treated is heart failure or acute heart failure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1A is a perspective view illustration depicting a balloon catheter system, according to some embodiments.

FIG. 1B is a perspective view illustration depicting the balloon catheter system of FIG. 1A having balloons in an inflated configuration, according to some embodiments.

FIG. 1C is a side view schematic of a balloon catheter system, according to some embodiments.

FIGS. 2A-2B are illustrations depicting cross sections of a first balloon catheter of a balloon catheter system, according to some embodiments.

FIG. 3A is an illustration depicting a side view of the elongate body of a first balloon catheter, having an inlet for receiving a third catheter into a third lumen and an outlet allowing exit of the third catheter from the third lumen, FIG. 3B is a side view of a first balloon catheter, FIG. 3C is a perspective view of the balloon and distal tip of a first balloon catheter, and FIG. 3D is a side view of a connector for a first balloon catheter, all according to some embodiments.

FIG. 4A is a perspective view illustration depicting a schematic of a second balloon catheter of a balloon catheter system, FIG. 4B is a side view of a second balloon catheter, FIG. 4C is a perspective view of the balloon and distal tip of a second balloon catheter, FIG. 4D is a cross-sectional view of a second balloon catheter, and FIG. 4E is a side view of a connector for a second balloon catheter, according to some embodiments.

FIG. 5 is a front view illustration depicting a third balloon catheter of a balloon catheter system, according to some embodiments.

FIG. 6 is a front view illustration depicting a detachable sheath for inserting a balloon catheter system, according to some embodiments.

FIG. 7 is a schematic depicting a balloon catheter system for the treatment of heart failure, according to some embodiments.

FIGS. 8A-8D are a series of section view illustrations depicting a method for using the balloon catheter systems described herein to treat heart failure, according to some embodiments.

FIGS. 9A-9D are a series of section view illustrations depicting a method for advancing a pulmonary artery catheter to the third target location at the pulmonary artery.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Unless otherwise defined, all technical terms used herein have the same meaning as commonly understood by one of ordinary skill in the art to which this disclosure belongs. As used herein, the singular forms “a,” “an,” and “the” include plural references unless the context clearly indicates otherwise. Any reference to “or” herein is intended to encompass “and/or” unless otherwise stated. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than,” “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The present disclosure describes balloon catheter systems and methods for the treatment of heart failure. Heart failure is typically accompanied by autonomic dysregulation which is characterized by enhanced sympathetic activity and withdrawal of parasympathetic control. Autonomic dysregulation has long been recognized as an important mediator of increased mortality and morbidity in myocardial infarction and heart failure. The sustained increase of sympathetic drive along with reduced parasympathetic activity and activation of the renin-angiotensin-aldosterone system (“RAAS”) in heart failure also contribute to progressive left ventricular dysfunction, progressive left ventricular remodeling, end-organ damage and ultimately death. The mechanisms responsible for sustained sympathetic excitation in heart failure are not fully understood. It is generally believed that the arterial reflexes, including the carotid sinus baroreflex, that are normally inhibitory to this system, have reduced sensitivity in heart failure and, therefore, allow sympathetic outflow to proceed unchecked. Several studies have shown an abnormally depressed arterial baroreflex control in heart failure. This autonomic maladaptation in heart failure can lead to increased heart rate, dysregulation of key components of the cardiac beta-adrenergic receptor signal transduction pathway, dysregulation of nitric oxide signaling and the development of life-threatening ventricular arrhythmias. The balloon catheter system and methods of the present disclosure promote baroreceptor activation which may reduce sympathetic overdrive and augment parasympathetic control causing reduce heart rate, improve adrenergic receptor and nitric oxide signaling, increase the threshold for lethal ventricular arrhythmias, improve global left ventricular function, and partially reverse left ventricular chamber remodeling.

### Catheter Systems and Devices

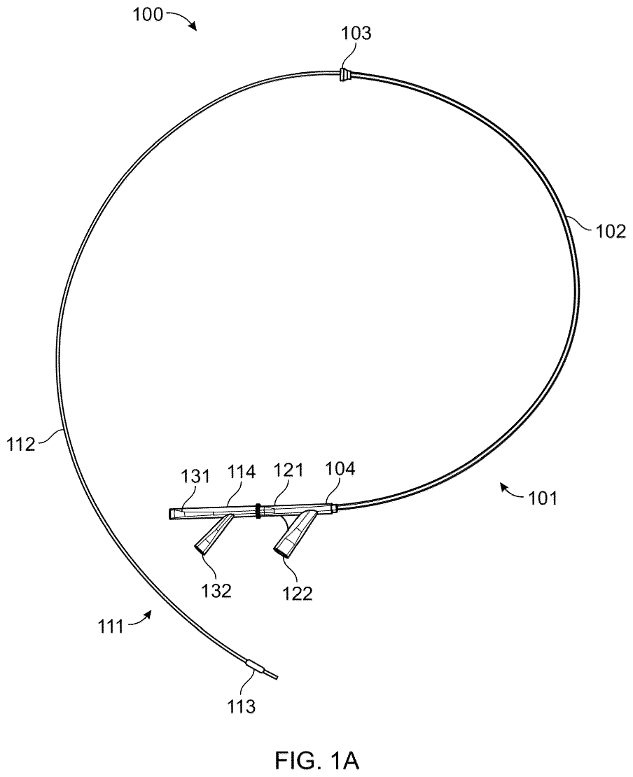

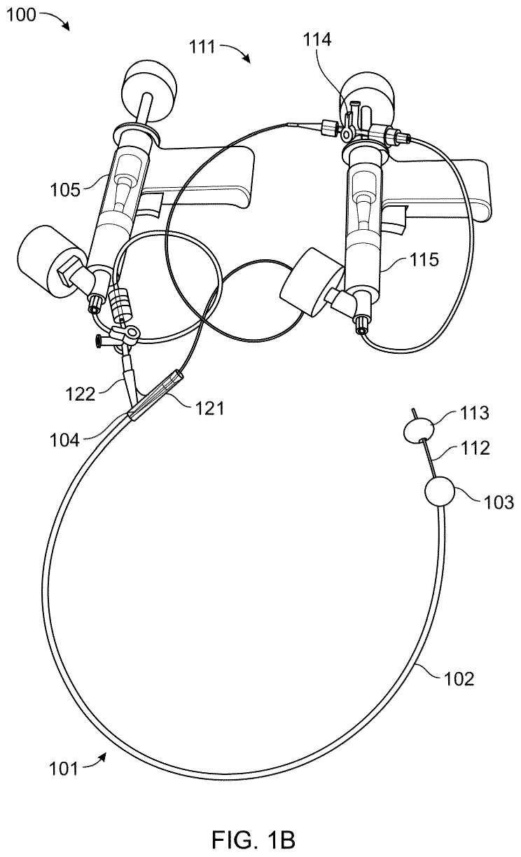

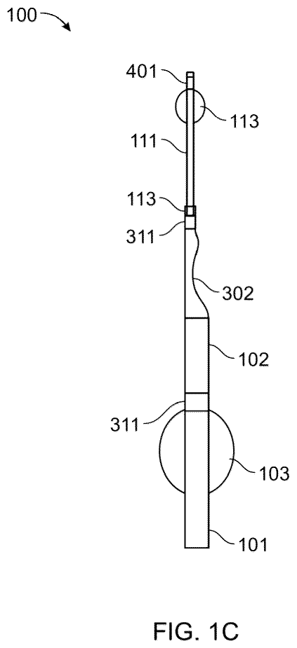

As described herein, the present disclosure provides a balloon catheter system for the treatment of heart failure. FIG. 1A is an illustration depicting a balloon catheter system 100, according to some embodiments.

Systemic venous congestion is one of the hallmarks of the syndrome of heart failure. In some cases, venous congestion results in impaired renal perfusion and activation of the renin-angiotensin-aldosterone (“RAAS”) system, and both contribute to renal dysfunction in heart failure patients. In addition, blood and lymphatic return from the lower extremities and abdomen may be impeded by higher venous pressures. The balloon catheter system 100 may include a first balloon catheter 101 configured to form a low-pressure area at or below the renal vein improving blood and lymphatic return and organ function. The first balloon catheter 101 may further stimulate the vagus nerve causing enhanced blood accommodation in the lower extremities and abdomen.

The first balloon catheter 101 may comprise an elongate body 102 and a first balloon 103 positioned at the distal end of the elongate body 102. The first balloon catheter 101 may be configured to be inserted into the femoral vein of a subject such that the first balloon 103 may be advanced to a first target location at the inferior vena cava (“IVC”) proximal to either the left or right renal veins. The first balloon catheter 101 may further comprise a first Y-connector 104 having a first port 121 configured to receive a second balloon catheter 111 therethrough and a second port 122 for attaching a pump. Upon placement of the first balloon 103 at the first target location, the first balloon 103 may be inflated using the second port 122 of the first Y-connector 104. FIG. 1B is an illustration depicting a balloon catheter system 100 having balloons in an inflated configuration, according to some embodiments. The first balloon 103 may be inflated by connecting a first pump 105, such as, for example, a syringe pump, to the second port 122 of the first Y-connector 104. The first pump 105 may either be used to inflate or deflate the first balloon 103. The first balloon 103 of the first balloon catheter 101 may comprise a multi-layered construction to prevent rupture of the balloon when inflated.

In some embodiments, the first balloon catheter 101 is a disposable single use device that is made of a biocompatible material. In some embodiments, the elongate body 102 of the first balloon catheter 101 comprises a material selected the group consisting of polyethylene terephthalate, polyethylene, polyamide, or combinations thereof. In some embodiments, the first balloon 103 of the first balloon catheter 101 is made of a silica and/or nylon material. In some embodiments, the first balloon 103 of the first balloon catheter 101 is made of a polyolefin material. In some embodiments, the first balloon 103 of the first balloon catheter 101 is made of polyvinyl chloride. In some embodiments, the elongate body 102 is provided with one or more of a hydrophilic coating, an anti-coagulation coating, an anti-condensation coating, or a lubricating coating. The coating may be made of polyvinylpyrrolidone (PVP), for example.

In some embodiments, the elongate body 102 of the first balloon catheter 101 may have a length of at least about 5 centimeters (“cm”), 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, 100 cm, 105 cm, 110 cm, or any values there between. In some embodiments, the elongate body 102 of the first balloon catheter 101 may have a length of at most about 110 cm, 105 cm, 100 cm, 95 cm, 90 cm, 85 cm, 80 cm, 75 cm, 70 cm, 65 cm, 60 cm, 55 cm, 50 cm, 45 cm, 40 cm, 35 cm, 30 cm, 25 cm, 20 cm, 15 cm, 10 cm, 5 cm, or any values therebetween. In some embodiments, the elongate body 102 of the first balloon catheter 101 may have a length from about 40 cm to about 110 cm.

In some embodiments, the elongate body 102 of the first balloon catheter 101 may have outer diameter of at least about 2 French (“Fr”), 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the elongate body 102 of the first balloon catheter 101 may have an outer diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the elongate body 102 of the first balloon catheter 101 may have outer diameter from about 10 Fr to about 20 Fr. In some embodiments, the elongate body 102 of the first balloon catheter 101 may have outer diameter from about 13 Fr to about 17 Fr. In some embodiments, the elongate body 102 of the first balloon catheter 101 may have outer diameter of about 16 Fr. In some embodiments, the elongate body 102 of the first balloon catheter 101 may have outer diameter of about 15 Fr.

In some embodiments, the first balloon 103 of the first balloon catheter 101 may have a length of at least about 1 millimeters (“mm”), 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or any values therebetween. In some embodiments, the first balloon 103 of the first balloon catheter 101 may have a length of at most about 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some embodiments, the first balloon 103 of the first balloon catheter 101 may have a length from about 5 mm to about 20 mm. In some embodiments, the first balloon 103 of the first balloon catheter 101 may have a length from about 5 mm to about 15 mm. In some embodiments, the first balloon 103 of the first balloon catheter 101 may have a length of about 5 mm. In some embodiments, the first balloon 103 of the first balloon catheter 101 may have a length of about 6 mm. In some embodiments, the first balloon 103 of the first balloon catheter 101 may have a length of about 7 mm. In some embodiments, the first balloon 103 of the first balloon catheter 101 may have a length of about 8 mm. In some embodiments, the first balloon 103 of the first balloon catheter 101 may have a length of about 9 mm. In some embodiments, the first balloon 103 of the first balloon catheter 101 may have a length of about 10 mm.

In some embodiments, the first balloon 103 of the first balloon catheter 101 may have an inflated diameter of at least about 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, or any values there between. In some embodiments, the first balloon 103 of the first balloon catheter 101 may have an inflated diameter of at most about 40 mm, 39 mm, 38 mm, 37 mm, 36 mm, 35 mm, 34 mm, 33 mm, 32 mm, 31 mm, 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, or any values therebetween. In some embodiments, the first balloon 103 of the first balloon catheter 101 may have an inflated diameter from about 15 mm to about 35 mm. In some embodiments, the first balloon 103 of the first balloon catheter 101 may have an inflated diameter from about 15 mm to about 30 mm. In some embodiments, the first balloon 103 of the first balloon catheter 101 may have an inflated diameter from about 18 mm to about 25 mm.

In some embodiments, the first balloon 103 of the first balloon catheter 101 may be inflated to a pressure of at least about 0.1 atm, 0.2 atm, 0.3 atm, 0.4 atm, 0.5 atm, 0.6 atm, 0.7, 0.8 atm, 0.9 atm, 1 atm, 2 atm, 3 atm, 4 atm, 5 atm, 6 atm, 7 atm, 9 atm, 10 atm, or any values therebetween. In some embodiments, the first balloon 103 of the first balloon catheter 101 may be inflated to a pressure of at most about 10 atm, 9 atm, 8 atm, 7 atm, 6 atm, 5 atm, 4 atm, 3 atm, 2 atm, 1 atm, 0.9 atm, 0.8 atm, 0.7 atm, 0.6 atm, 0.5 atm, 0.4 atm, 0.3 atm, 0.2 atm, 0.1 atm, or any values therebetween. In some embodiments, the first balloon 103 of the first balloon catheter 101 may be inflated to a pressure from about 0.1 atm to about 2 mm. In some embodiments, the first balloon 103 of the first balloon catheter 101 may be inflated to a pressure from about 1 atm to about 2 mm.

In some embodiments, the first balloon 103 of the first balloon catheter 101 is made of a highly compliant material. In some embodiments, the first balloon 103 of the first balloon catheter 101 may have a fatigue strength (i.e., amplitude or range of cyclic stress that can be applied to the material without causing fatigue failure) of at least 100, 150, 200, 250, 300, 350, 400, 450, 500, or more cycles of inflation and deflation.

The balloon catheter system 100 may further include a second balloon catheter 111 configured to form a low-pressure area near the left internal jugular vein and/or thoracic duct, improving return to the superior vena cava (“SVC”) and the right atrium of the heart. The second balloon catheter 111 may further be configured to stimulate the vagus nerve causing improved cardiac remodeling and function. Stimulation of the vagus nerve may cause vasodilation which can increase blood accommodation in the venous bed and reduces heart preload.

The second balloon catheter 111 may comprise an elongate body 112 and a second balloon 113 positioned at the distal end of the elongate body 112. The second balloon catheter 111 may further comprise a lumen for inflating the second balloon 113. The second balloon catheter 111 may be configured to be inserted into the first port 121 of the first Y-connector 104 such that it may be advanced through a lumen in the first balloon catheter 101 past the IVC and onto a second target location proximal to either the left or right subclavian vein. Upon placement of the second balloon 113 at the second target location, the second balloon 113 may be inflated using a second Y-connector 114. The second Y-connector 114 may comprise a first port 131 for receiving a guidewire and a second port 132 for attaching a pump. The second balloon 113 may be inflated by a connecting a second pump 115, such as, for example, a syringe pump, to the second port 132 of the second Y-connector 114. The second pump 115 may either be used to inflate or deflate the second balloon 113. The second balloon 113 of the second balloon catheter 111 may comprise a multi-layered construction to prevent rupture of the balloon when inflated.

In some embodiments, the second balloon catheter 111 is a disposable single use device that is made of a biocompatible material. In some embodiments, the elongate body 112 of the second balloon catheter 111 polyvinyl chloride. In some embodiments, the second balloon 113 of the second balloon catheter 111 is made of polyvinyl chloride. In some embodiments the second balloon 113 of the second balloon catheter 111 is made of a silica and/or nylon material. In some embodiments, the second balloon 113 of the second balloon catheter 111 is made of a polyolefin material. In some embodiments, the elongate body 112 is provided with one or more of a hydrophilic coating, an anti-coagulation coating, an anti-condensation coating, or a lubricating coating. The coating may be made of polyvinylpyrrolidone (PVP), for example.

In some embodiments, the elongate body 112 of the second balloon catheter 111 may have a length of at least about 5 cm, 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, 100 cm, 105 cm, 110 cm, 115 cm, 120 cm, 125 cm, 130 cm, 135 cm, 140 cm, 145 cm, 150 cm, 155 cm, 160 cm, 165 cm, 170 cm, 175 cm, 180 cm, 185 cm, 190 cm, 195 cm, 200 cm or any values there between. In some embodiments, the elongate body 112 of the second balloon catheter 111 may have a length of at most about 200 cm, 195 cm, 190 cm, 185 cm, 180 cm, 175 cm, 170 cm, 165 cm, 160 cm, 155 cm, 150 cm, 145 cm, 140 cm, 135 cm, 130 cm, 125 cm, 120 cm, 115 cm, 110 cm, 105 cm, 100 cm, 95 cm, 90 cm, 85 cm, 80 cm, 75 cm, 70 cm, 65 cm, 60 cm, 55 cm, 50 cm, 45 cm, 40 cm, 35 cm, 30 cm, 25 cm, 20 cm, 15 cm, 10 cm, 5 cm, or any values therebetween. In some embodiments, the elongate body 112 of the second balloon catheter 111 may have a length from about 50 cm to about 100 cm. In some embodiments, the elongate body 112 of the second balloon catheter 111 may have a length from about 70 cm to about 90 cm. In some embodiments, the elongate body 112 of the second balloon catheter 111 may have a length of about 80 cm.

In some embodiments, the elongate body 112 of the second balloon catheter 111 may have an outer diameter of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the elongate body 112 of the second balloon catheter 111 may have an outer diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the elongate body 112 of the second balloon catheter 111 may have outer diameter from about 2 Fr and about 12 Fr. In some embodiments, the elongate body 112 of the second balloon catheter 111 may have outer diameter from about 4 Fr and about 10 Fr. In some embodiments, the elongate body 112 of the second balloon catheter 111 may have outer diameter of about 6 Fr.

In some embodiments, the second balloon 113 of the second balloon catheter 111 may have a length of at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or any values therebetween. In some embodiments, the second balloon 113 of the second balloon catheter 111 may have a length of at most about 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some embodiments, the second balloon 113 of the second balloon catheter 111 may have a length from about 5 mm to about 20 mm. In some embodiments, the second balloon 113 of the second balloon catheter 111 may have a length from about 5 mm to about 15 mm. In some embodiments, the second balloon 113 of the second balloon catheter 111 may have a length of about 10 mm.

In some embodiments, the second balloon 113 of the second balloon catheter 111 may have an inflated diameter of at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or any values there between. In some embodiments, the second balloon 113 of the second balloon catheter 111 may have an inflated diameter of at most about 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some embodiments, the second balloon 113 of the second balloon catheter 111 may have an inflated diameter from about 1 mm to about 20 mm. In some embodiments, the second balloon 113 of the second balloon catheter 111 may have an inflated diameter from about 5 mm to about 15 mm. In some embodiments, the second balloon 113 of the second balloon catheter 111 may have an inflated diameter from about 8 mm to about 10 mm.

In some embodiments, the second balloon 113 of the second balloon catheter 111 may be inflated to a pressure of at least about 0.1 atm, 0.2 atm, 0.3 atm, 0.4 atm, 0.5 atm, 0.6 atm, 0.7, 0.8 atm, 0.9 atm, 1 atm, 2 atm, 3 atm, 4 atm, 5 atm, 6 atm, 7 atm, 9 atm, 10 atm, or any values therebetween. In some embodiments, the second balloon 113 of the second balloon catheter 111 may be inflated to a pressure of at most about 10 atm, 9 atm, 8 atm, 7 atm, 6 atm, 5 atm, 4 atm, 3 atm, 2 atm, 1 atm, 0.9 atm, 0.8 atm, 0.7 atm, 0.6 atm, 0.5 atm, 0.4 atm, 0.3 atm, 0.2 atm, 0.1 atm, or any values therebetween. In some embodiments, the second balloon 113 of the second balloon catheter 111 may be inflated to a pressure from about 0.1 atm to about 2 mm. In some embodiments, the second balloon 113 of the second balloon catheter 111 may be inflated to a pressure from about 1 atm to about 2 mm.

In some embodiments, the second balloon 113 of the second balloon catheter 111 is made of a highly compliant material. In some embodiments, the second balloon 113 of the second balloon catheter 111 may have a fatigue strength of at least 100, 150, 200, 250, 300, 350, 400, 450, 500, or more cycles of inflation and deflation.

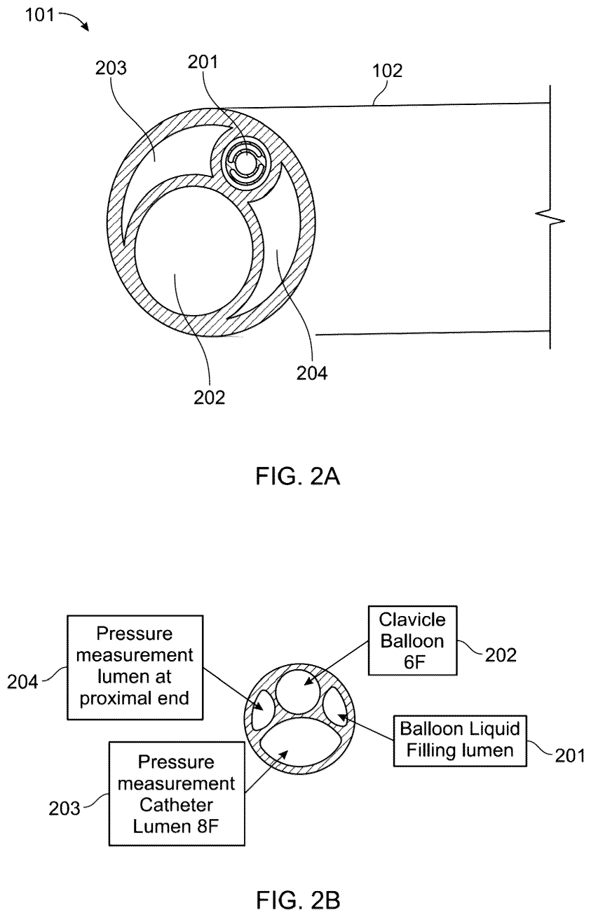

FIGS. 2A-2B are an illustration depicting a cross section of the first balloon catheter 101 of the balloon catheter system 100, according to some embodiments. FIGS. 2A and 2B show alternate configurations of lumens. In some embodiments, the first balloon catheter 101 comprises a plurality of lumens. In some embodiments, the first balloon catheter 101 comprises at least 2 lumens. In some embodiments, the first balloon catheter 101 comprises at least 3 lumens. In some embodiments, the first balloon catheter 101 comprises at least 4 lumens. In some embodiments, the first balloon catheter 101 comprises at least 5 lumens. In some embodiments, the first balloon catheter 101 comprises at least 6 lumens.

The first balloon catheter 101 may comprise a first lumen 201 configured to inflate the first balloon 103. In some embodiments, the first lumen 201 of the first balloon catheter 101 may have an inner diameter of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the first lumen 201 of the first balloon catheter 101 may have an inner diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the first lumen 201 of the first balloon catheter 101 may have an inner diameter from about 2 Fr and about 10 Fr. In some embodiments, the first lumen 201 of the first balloon catheter 101 may have an inner diameter from about 2 Fr and about 6 Fr. In some embodiments, the first lumen 201 of the first balloon catheter 101 may have an inner diameter of about 2 Fr.

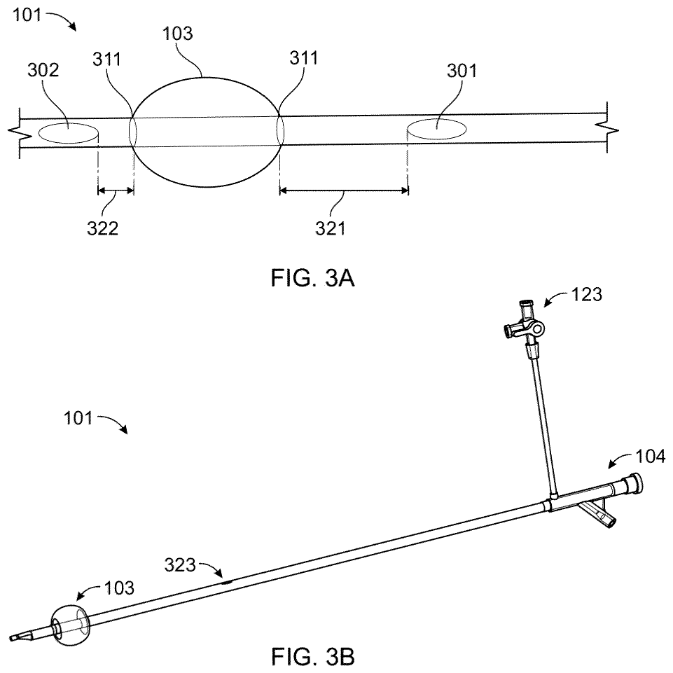

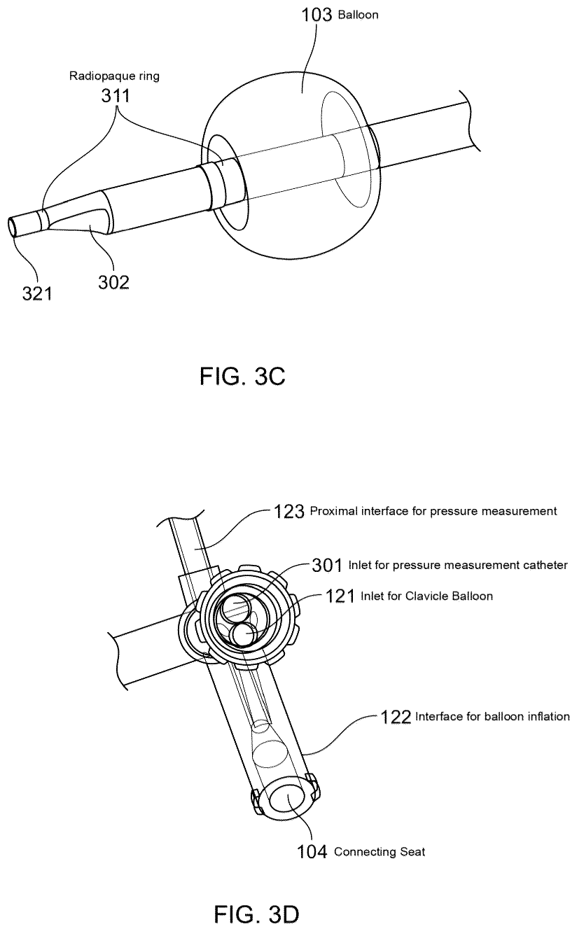

The first balloon catheter 101 may comprise a second lumen 202 configured to allow advancement of the second balloon catheter 111 therethrough. In some embodiments, the second lumen 202 of the first balloon catheter 101 may have an inner diameter of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the second lumen 202 of the first balloon catheter 101 may have an inner diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the second lumen 202 of the first balloon catheter 101 may have an inner diameter from about 2 Fr and about 10 Fr. In some embodiments, the second lumen 202 of the first balloon catheter 101 may have an inner diameter from about 4 Fr and about 8 Fr. In some embodiments, the second lumen 202 of the first balloon catheter 101 may have an inner diameter of about 7 Fr. In some embodiments, the second lumen 202 terminates at a distal end or tip of the first balloon catheter 101 at a distal end port 321 from where the second balloon catheter 111 may be advanced out of (see FIGS. 1C and 3C).

The first balloon catheter 101 may comprise a third lumen 203 configured to allow advancement of a third catheter therethrough. In some embodiments, the third lumen 203 of the first balloon catheter 101 may have an inner diameter of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the third lumen 203 of the first balloon catheter 101 may have an inner diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the third lumen 203 of the first balloon catheter 101 may have an inner diameter from about 2 Fr and about 10 Fr. In some embodiments, the third lumen 203 of the first balloon catheter 101 may have an inner diameter from about 4 Fr and about 8 Fr. In some embodiments, the third lumen 203 of the first balloon catheter 101 may have inner diameter of about 7 Fr.

The first balloon catheter 101 may comprise a fourth lumen 204 which may be in fluid communication with a port 323 on the body of the first balloon catheter 101 to serve as a pressure detection line (see FIG. 3B). For example, the port 323 may be located proximal to the first balloon 103 by about 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm, so that pressure at the inferior vena cava (“IVC”) and/or femoral vein(s) may be measured. In some embodiments, the fourth lumen 204 of the first balloon catheter 101 may have an inner diameter of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the fourth lumen 204 of the first balloon catheter 101 may have an inner diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the fourth lumen 204 of the first balloon catheter 101 may have an inner diameter from about 2 Fr and about 10 Fr. In some embodiments, the fourth lumen 204 of the first balloon catheter 101 may have an inner diameter from about 4 Fr and about 8 Fr. In some embodiments, the fourth lumen 204 of the first balloon catheter 101 may have inner diameter of about 7 Fr.

FIGS. 3A-3D are illustrations depicting the elongate body 102 of the first balloon catheter 101, having an inlet 301 for receiving a third catheter into the third lumen 203 and an outlet 302 allowing exit of the third catheter from the third lumen 203 (see FIGS. 3A and 3C), according to some embodiments. The inlet 301 may be positioned at the proximal end (e.g., the end closest to a user of the balloon catheter system 100) of the elongate body 102 of the first balloon catheter 101, as shown for example at FIG. 3A. In some embodiments, the inlet distance 321 (e.g., the between the inlet 301 and first balloon 103) is at least about 1 cm, 2 cm, 4 cm, 6 cm, 8 cm, 10 cm, 12 cm, 14 cm, 16 cm, 18 cm, 20 cm, 22 cm, 24 cm, 26 cm, 28 cm, 30 cm, or any values there between. In some embodiments, the inlet distance 321 is at most about 30 cm, 28 cm, 26 cm, 24 cm, 22 cm, 20 cm, 18 cm, 16 cm, 14 cm, 12 cm, 10 cm, 8 cm, 6 cm, 4 cm, 2 cm, 1 cm, or any values therebetween. In some embodiments, the inlet distance 321 is from about 4 cm to about 18 cm. In some embodiments, the inlet distance 321 is from about 6 cm to about 14 cm. In some embodiments, the inlet distance 321 is from about 8 cm to about 12 cm. In some embodiments, the inlet distance 321 is about 10 cm. In some embodiments, as shown in FIGS. 3B and 3D, the inlet 301 is positioned at the Y-connector 104. The Y-connector 104 may also connect to a further connector (e.g., a Luer connector) including a port 123 to be in fluid communication to the port 323 via fourth lumen 204 for pressure measurement at the inferior vena cava and/or femoral vein(s).

In some embodiments, the inlet 301 of the first balloon catheter 101 may have a diameter of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the inlet 301 of the first balloon catheter 101 may have a diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the inlet 301 of the first balloon catheter 101 may have a diameter from about 2 Fr and about 10 Fr. In some embodiments, the inlet 301 of the first balloon catheter 101 may have a diameter from about 4 Fr and about 8 Fr. In some embodiments, the inlet 301 of the first balloon catheter 101 may have a diameter of about 7 Fr.

The outlet 302 may be positioned at the distal end (e.g., the end farthest from a user of the balloon catheter system 100) of the elongate body 102 of the first balloon catheter 101. In some embodiments, the outlet 302 may be positioned at a lateral side wall of the elongate body 102 proximal to the distal end. In some embodiments, the outlet distance 322 (e.g., the distance between the outlet 302 and first balloon 103) is at least about 0.1 cm, 0.2 cm, 0.3 cm, 0.4 cm, 0.5 cm, 0.6 cm, 0.7 cm, 0.8 cm, 0.9 cm, 1 cm, 2 cm, 3 cm, 4 cm, 5 cm, 6 cm, 7 cm, 8 cm, 9 cm, 10 cm, or any values there between. In some embodiments, the outlet distance 322 is at most about 10 cm, 9 cm, 8 cm, 7 cm, 6 cm, 5 cm, 4 cm, 3 cm, 2 cm, 1 cm, 0.9 cm, 0.8 cm, 0.7 cm, 0.6 cm, 0.5 cm, 0.4 cm, 0.3 cm, 0.2 cm, 0.1 cm, or any values therebetween. In some embodiments, the outlet distance 322 is from about 0.1 cm to about 5 cm. In some embodiments, the outlet distance 322 is from about 0.2 cm to about 4 cm. In some embodiments, the outlet distance 322 is from about 0.4 cm to about 2 cm. In some embodiments, the outlet distance 321 is about 1 cm.

In some embodiments, the outlet 302 of the first balloon catheter 101 may have a diameter of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the outlet 302 of the first balloon catheter 101 may have a diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the outlet 302 of the first balloon catheter 101 may have a diameter from about 2 Fr and about 10 Fr. In some embodiments, the out 302 of the first balloon catheter 101 may have a diameter from about 4 Fr and about 8 Fr. In some embodiments, the outlet 302 of the first balloon catheter 101 may have a diameter of about 7 Fr.

The first balloon 103 of the first balloon catheter 101 may further at least one radio-opaque marker 311 allowing a user to visualize the first balloon 103 under radiography or fluoroscopy, as shown in FIG. 1C. In some embodiments, the radio-opaque markers 311 are metal markers. In some embodiments, the first balloon 101 comprises two radio-opaque markers 311. In some embodiments, the radio-opaque marker(s) 311 are positioned at one or more of the proximal or distal ends of the first balloon 103. In some embodiments, as shown in FIG. 1C, the radio-opaque markers 311 are positioned at or near the first balloon 103 and at or near the outlet port 302 for the third balloon catheter 501. In some embodiments, there may be a radio-opaque marker 311 located at or near a distal end or tip of the first balloon catheter 101. In some embodiments, the radio-opaque marker(s) 311 are in the form of a ring, such as a thin-walled ring. In some embodiments, the radio-opaque marker(s) 311 are made of a radio-opaque material including but not limited to platinum, platinum iridium, gold, and alloys thereof. In some embodiments, the radio-opaque markers are used to guide any one of the first balloon through the vasculature of the patient to a target location at or near the renal vein.

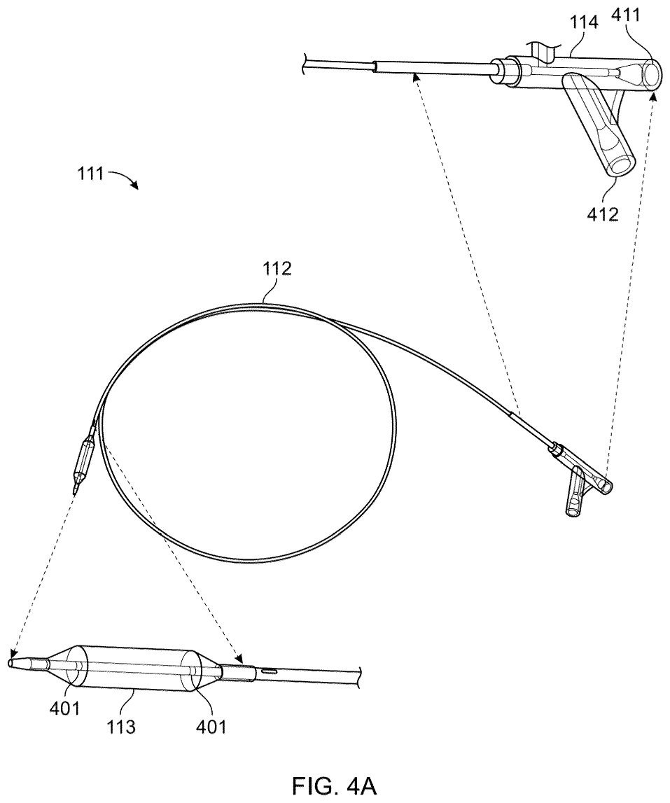

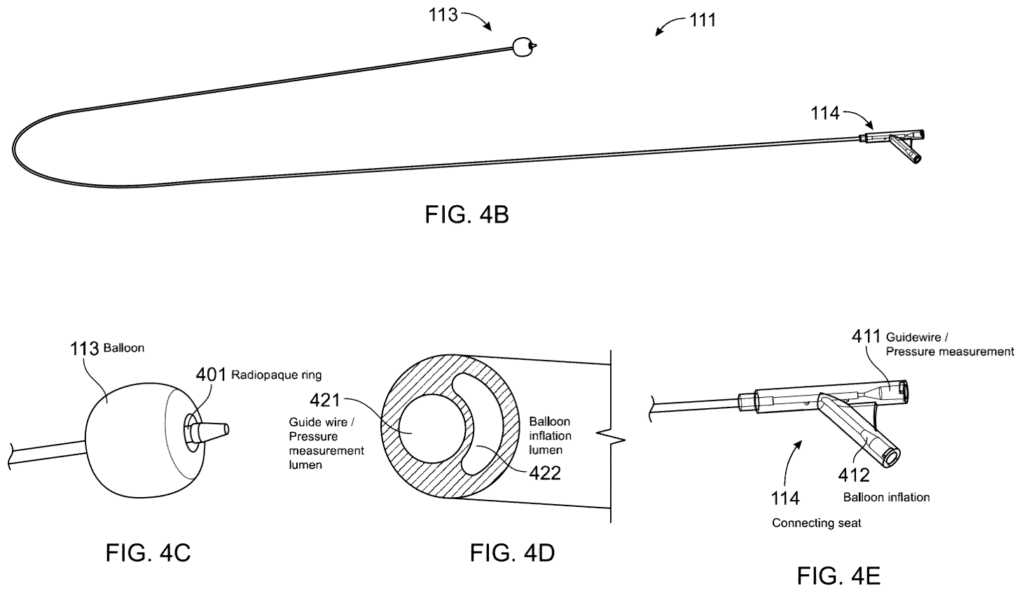

FIGS. 4A-4E are illustrations depicting the second balloon catheter 111 of the balloon catheter system 100, according to some embodiments. The second balloon catheter 111, may further comprise a lumen for advancing a guidewire. The guidewire may facilitate advancement of the second balloon 113 to the second target location. The guidewire, according to one aspect, may be formed of a flexible material to accommodate anatomical complications such as complex and tortuous vasculature. The second y-connector 114 of the second balloon catheter 111 may comprise a first port 411 for inserting the guidewire into lumen. The second Y-connector 114 of the second balloon catheter 111 may comprise a second port 412 for connecting a pump, (e.g., a syringe pump) for inflating the second balloon 113. The second balloon catheter 111 may further comprise a lumen 421 for measuring the subclavian pressure configured to be connected to a pressure measuring device. The lumen 412 may be in fluid communication with the first port 411. The second balloon catheter 111 may further comprise a lumen 422 for inflating the second balloon 113. The lumen 422 may be in fluid communication with the second port 412. The pressure measuring device may continuously or discretely measure the pressure at an area near second balloon 113 (e.g., the subclavian pressure).

The second balloon 113 of the second balloon catheter 111 may further at least one radio-opaque marker 401 allowing a user to visualize the second balloon 113 under radiography or fluoroscopy, as shown in FIG. 1C. In some embodiments, the radio-opaque markers 401 are metal markers. In some embodiments, the second balloon 113 comprises two radio-opaque markers 401. In some embodiments, the radio-opaque marker(s) 401 are positioned at one or more of the proximal or distal ends of the second balloon 113. In some embodiments, the radio-opaque marker(s) 401 are in the form of a ring, such as a thin-walled ring. In some embodiments, the radio-opaque marker(s) 401 are made of a radio-opaque material including but not limited to platinum, platinum iridium, gold, and alloys thereof. In some embodiments, the radio-opaque markers are used to guide any one of the first balloon through the vasculature of the patient to a target location at or near the subclavian vein.

FIG. 5 is an illustration depicting the third balloon catheter 501 of the balloon catheter system 100, according to some embodiments. Heart failure is commonly associated with increased pulmonary arterial pressure (“PAP”) and pulmonary capillary wedge pressure (“PCWP”), therefore treating heart failure requires monitoring of hemodynamics. The third balloon catheter 501, may be used to measure hemodynamics during treatment of heart failure.

The third balloon catheter 501 may be a floating balloon catheter. The third balloon catheter 501 may be a pulmonary artery catheter (“PAC”), such as, for example a Swan-Ganz catheter, or any other catheter that allows for hemodynamic monitoring. The third balloon catheter 501 may comprise an elongate body 502 and a third balloon 503 positioned at the distal end of the elongate body 502. The third balloon catheter may further comprise a lumen for inflating the third balloon 503. The third balloon catheter 501 may be configured be inserted into the inlet 301 of the first balloon catheter 101 such that it may be advanced through the third lumen 203 and through the outlet 302 onto a third target location proximal to the pulmonary artery. The third balloon catheter 501 may further comprise a lumen configured to receive a guidewire therethrough. The guidewire may facilitate advancement of the third balloon 503 to the third target location. The guidewire, according to one aspect, is formed of a flexible material to accommodate anatomical complications such as complex and tortuous vasculature. Upon placement of the third balloon 503 at the third target location, the third balloon 503 may be inflated using the inflation line 504. The third balloon 503 may be inflated by a connecting a third pump 505, such as, for example, a syringe pump, to the inflation line 504. The third pump 505 may either be used to inflate or deflate the third balloon 503. The third balloon 503 of the third balloon catheter 501 may comprise a multi-layered construction to prevent rupture of the balloon when inflated.

The third balloon catheter 501 may further comprise a lumen for measuring the pulmonary artery pressure configured to be connected to a distal pressure line 506. The distal pressure line 506 may be configured to be connected to a pressure measuring device which continuously or discretely measures the pressure at the third balloon 503 (e.g., the pulmonary artery pressure). The third balloon catheter 501 may further comprise a lumen for measuring the femoral vein pressure having an opening 508 at the proximal end (e.g., the end closest to a user of the balloon catheter system 100) of the third balloon catheter 501. During operation of the balloon catheter system 100, the third balloon catheter 501 is advanced through the first balloon catheter via the inlet 301, such that the opening 508 is located near femoral vein. The lumen for measuring the femoral vein pressure may be configured to be connected to a proximal pressure line 507. The proximal pressure line 507 may be configured to be connected to a pressure measuring device which continuously or discretely measures the pressure at the inlet 301 (e.g., the femoral vein pressure). The body of the third balloon catheter 501 may have one or more side ports and/or a distal to facilitate such pressure measurements. The third catheter may have separate lumens for inflation of the third balloon 503, pressure measurement at the pulmonary artery, pressure measurement at the inferior vena cava (for example, to measure central venous pressure) and/or femoral vein(s), and to facilitate advancement over a guidewire. The guidewire lumen may also serve as the lumen for pressure measurement at the pulmonary artery.

In some embodiments, the third balloon catheter 501 is a disposable single use device that is made of a biocompatible material. In some embodiments, the elongate body 502 of the third balloon catheter 501 is made of polyvinyl chloride. In some embodiments, the third balloon 503 of the third balloon catheter 501 is made of polyvinyl chloride. In some embodiments, the third balloon 503 of the third balloon catheter 501 is made of a latex material. In some embodiments, the elongate body 502 is provided with one or more of a hydrophilic coating, an anti-coagulation coating, an anti-condensation coating, or a lubricating coating. The coating may be made of polyvinylpyrrolidone (PVP), for example.

In some embodiments, the elongate body 502 of the third balloon catheter 501 may have a length of at least about 5 cm, 10 cm, 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, 100 cm, 105 cm, 110 cm, 115 cm, 120 cm, 125 cm, 130 cm, 135 cm, 140 cm, 145 cm, 150 cm, 155 cm, 160 cm, 165 cm, 170 cm, 175 cm, 180 cm, 185 cm, 190 cm, 195 cm, 200 cm, 205 cm, 210 cm, 215 cm, 220 cm, or any values there between. In some embodiments, the elongate body 502 of the third balloon catheter 501 may have a length of at most about 220 cm, 215 cm, 210 cm, 205 cm, 200 cm, 195 cm, 190 cm, 185 cm, 180 cm, 175 cm, 170 cm, 165 cm, 160 cm, 155 cm, 150 cm, 145 cm, 140 cm, 135 cm, 130 cm, 125 cm, 120 cm, 115 cm, 110 cm, 105 cm, 100 cm, 95 cm, 90 cm, 85 cm, 80 cm, 75 cm, 70 cm, 65 cm, 60 cm, 55 cm, 50 cm, 45 cm, 40 cm, 35 cm, 30 cm, 25 cm, 20 cm, 15 cm, 10 cm, 5 cm, or any values therebetween. In some embodiments, the elongate body 502 of the third balloon catheter 501 may have a length from about 50 cm to about 120 cm. In some embodiments, the elongate body 502 of the third balloon catheter 501 may have a length from about 70 cm to about 120 cm. In some embodiments, the elongate body 502 of the third balloon catheter 501 may have a length of about 110 cm.

In some embodiments, the elongate body 502 of the third balloon catheter 501 may have an outer diameter of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the elongate body 502 of the third balloon catheter 501 may have an outer diameter of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the elongate body 502 of the third balloon catheter 501 may have outer diameter from about 2 Fr and about 12 Fr. In some embodiments, the elongate body 502 of the third balloon catheter 501 may have outer diameter from about 4 Fr and about 10 Fr. In some embodiments, the elongate body 502 of the third balloon catheter 501 may have outer diameter of about 6 Fr.

In some embodiments, the third balloon 503 of the third balloon catheter 501 may have a length of at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or any values therebetween. In some embodiments, the third balloon 503 of the third balloon catheter 501 may have a length of at most about 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some embodiments, the third balloon 503 of the third balloon catheter 501 may have a length from about 1 mm to about 10 mm. In some embodiments, the third balloon 503 of the third balloon catheter 501 may have a length from about 3 mm to about 5 mm. In some embodiments, the third balloon 503 of the third balloon catheter 501 may have a length of about 3 mm. In some embodiments, the third balloon 503 of the third balloon catheter 501 may have a length of about 4 mm. In some embodiments, the third balloon 503 of the third balloon catheter 501 may have a length of about 5 mm.

In some embodiments, the third balloon 503 of the third balloon catheter 501 may have an inflated diameter of at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or any values there between. In some embodiments, the third balloon 503 of the third balloon catheter 501 may have an inflated diameter of at most about 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some embodiments, the third balloon 503 of the third balloon catheter 501 may have an inflated diameter from about 1 mm to about 20 mm. In some embodiments, the third balloon 503 of the third balloon catheter 501 may have an inflated diameter from about 1 mm to about 10 mm. In some embodiments, the third balloon 503 of the third balloon catheter 501 may have an inflated diameter from about 3 mm to about 5 mm. In some embodiments, the third balloon 503 of the third balloon catheter 501 may have an inflated diameter of about 3 mm. In some embodiments, the third balloon 503 of the third balloon catheter 501 may have an inflated diameter of about 4 mm. In some embodiments, the third balloon 503 of the third balloon catheter 501 may have an inflated diameter of about 5 mm.

In some embodiments, the third balloon 503 of the third balloon catheter 501 may be inflated to a pressure of at least about 0.1 atm, 0.2 atm, 0.3 atm, 0.4 atm, 0.5 atm, 0.6 atm, 0.7, 0.8 atm, 0.9 atm, 1 atm, 2 atm, 3 atm, 4 atm, 5 atm, 6 atm, 7 atm, 9 atm, 10 atm, or any values therebetween. In some embodiments, the third balloon 503 of the third balloon catheter 501 may be inflated to a pressure of at most about 10 atm, 9 atm, 8 atm, 7 atm, 6 atm, 5 atm, 4 atm, 3 atm, 2 atm, 1 atm, 0.9 atm, 0.8 atm, 0.7 atm, 0.6 atm, 0.5 atm, 0.4 atm, 0.3 atm, 0.2 atm, 0.1 atm, or any values therebetween. In some embodiments, the third balloon 503 of the third balloon catheter 501 may be inflated to a pressure from about 0.1 atm to about 2 mm. In some embodiments, the third balloon 503 of the third balloon catheter 501 may be inflated to a pressure from about 1 atm to about 2 mm.

In some embodiments, the third balloon 503 of the third balloon catheter 501 is made of a highly compliant material. In some embodiments, the third balloon 503 of the third balloon catheter 501 may have a fatigue strength of at least 100, 150, 200, 250, 300, 350, 400, 450, 500, or more cycles of inflation and deflation.

The third balloon 503 of the third balloon catheter 501 may further at least one radio-opaque marker allowing a user to visualize the third balloon 503 under radiography or fluoroscopy. In some embodiments, the radio-opaque markers are metal markers. In some embodiments, the radio-opaque marker(s) 501 are in the form of a ring, such as a thin-walled ring. In some embodiments, the radio-opaque marker(s) 501 are made of a radio-opaque material including but not limited to platinum, platinum iridium, gold, and alloys thereof. In some embodiments, the radio-opaque markers are used to guide any one of the first balloon through the vasculature of the patient to a target location at or near the pulmonary artery.

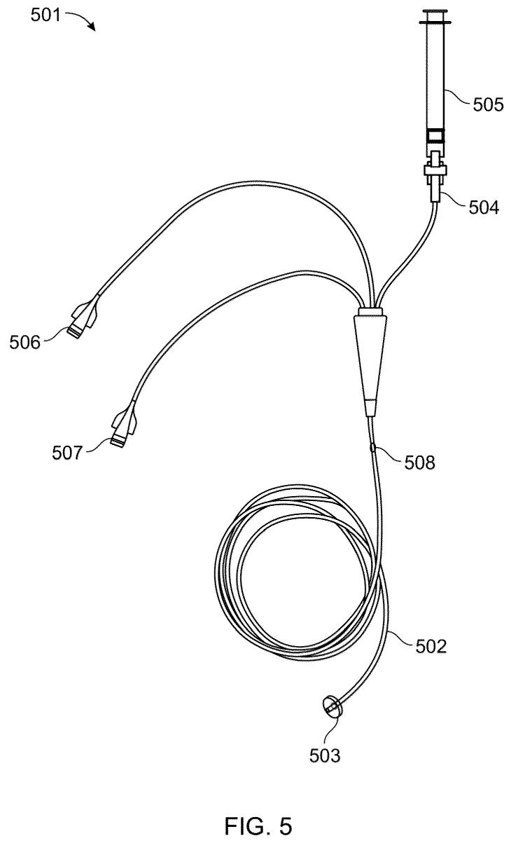

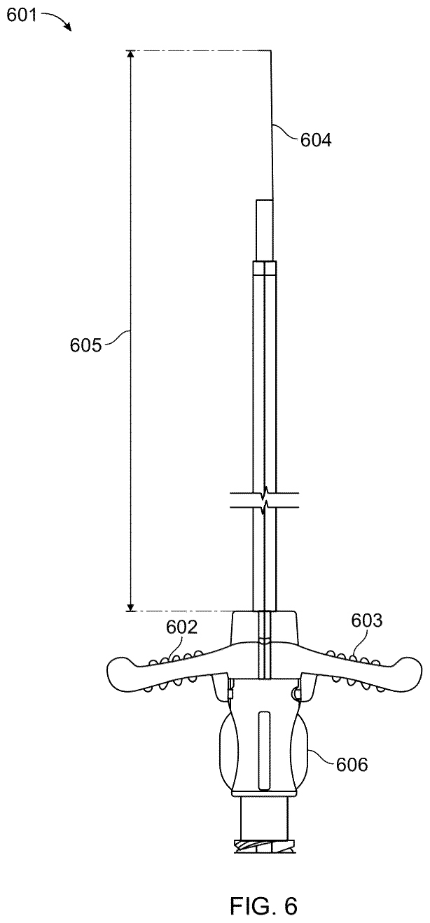

The balloon catheter system 100 may further include a tearable or detachable sheath puncture kit as well as consumable accessories. FIG. 6 is an illustration depicting a detachable sheath 601 for inserting the balloon catheter system 100, according to some embodiments. The detachable sheath 601 is configured to be inserted into a puncture site at the femoral vein of a subject. The detachable sheath 601 may comprise a first detachable arm 602 and a second detachable arm 603 that allow the detachable sheath to be torn and removed after placement of the first balloon 103 at the first target location.

The detachable sheath 601 may further comprise a tapered dilator 604 for enlarging the insertion site at the femoral vein. In some embodiments, the tapered dilator 604 may have a shaft length 605 of at least about 1 cm, 2 cm, 3 cm, 4 cm, 5 cm, 6 cm, 7 cm, 8 cm, 9 cm, 10 cm, 12 cm, 14 cm, 16 cm, 18 cm, 20 cm, 22 cm, 24 cm, 26 cm, 28 cm, 30 cm, 35 cm, 40 cm, 50 cm, or any values there between. In some embodiments, the tapered dilator 604 may have a shaft length 605 of at most about 50 cm, 40 cm, 35 cm, 30 cm, 28 cm, 26 cm, 24 cm, 22 cm, 20 cm, 18 cm, 16 cm, 14 cm, 12 cm, 10 cm, 9 cm, 8 cm, 7 cm, 6 cm, 5 cm, 4 cm, 3 cm, 2 cm, 1 cm, or any values therebetween. In some embodiments, the tapered dilator 604 may have a shaft length 605 from about 10 cm to about 50 cm. In some embodiments, the tapered dilator 604 may have a shaft length 605 from about 20 cm to about 40 cm. In some embodiments, the tapered dilator 604 may have a shaft length 605 of about 35 cm.

In some embodiments, the tapered dilator 604 has an outer diameter, at its widest point, of at least about 2 Fr, 3 Fr, 4 Fr, 5 Fr, 6 Fr, 7 Fr, 8 Fr, 9 Fr, 10 Fr, 11 Fr, 12 Fr, 13 Fr, 14 Fr, 15 Fr, 16 Fr, 17 Fr, 18 Fr, 19 Fr, 20 Fr, 21 Fr, 22 Fr, 24 Fr, 26 Fr, 28 Fr, 30 Fr, or any values therebetween. In some embodiments, the tapered dilator 604 has an outer diameter, at its widest point, of at most about 30 Fr, 28 Fr, 26 Fr, 24 Fr, 22 Fr, 20 Fr, 19 Fr, 18 Fr, 17 Fr, 16 Fr, 15 Fr, 14 Fr, 13 Fr, 12 Fr, 11 Fr, 10 Fr, 9 Fr, 8 Fr, 7 Fr, 6 Fr, 5 Fr, 4 Fr, 3 Fr, 2 Fr, or any values therebetween. In some embodiments, the tapered dilator 604 has an outer diameter, at its widest point, from about 10 Fr and about 20 Fr. In some embodiments, the tapered dilator 604 has an outer diameter, at its widest point, from about 14 Fr and about 20 Fr. In some embodiments, the tapered dilator 604 has an outer diameter, at its widest point, of about 16 Fr.

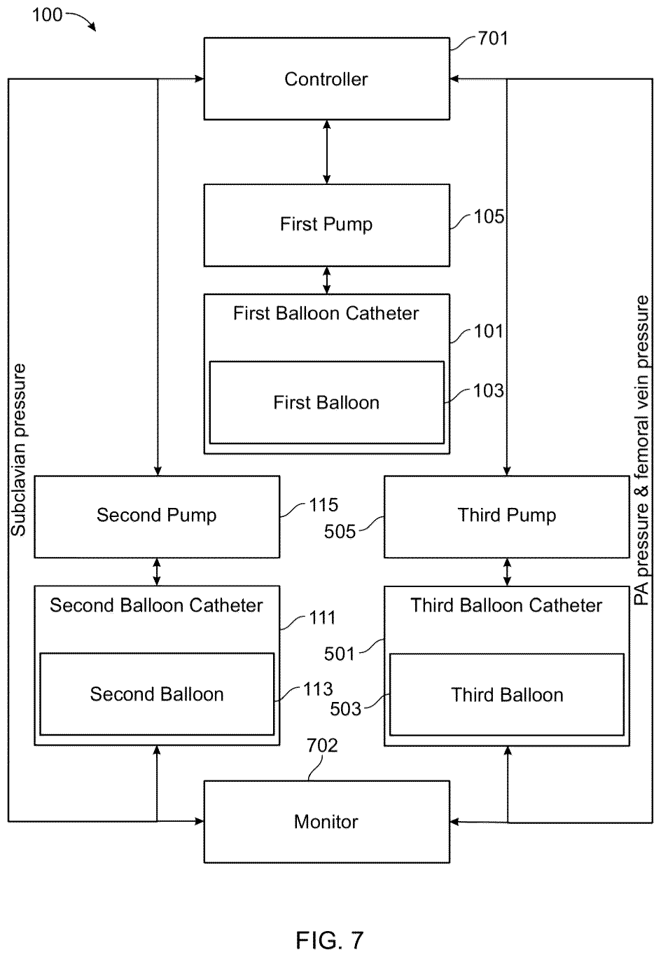

FIG. 7 is a schematic depicting the balloon catheter system 100 for the treatment of heart failure, according to some embodiments. The first balloon catheter 101 may be mechanically connected to a first pump 105 configured to inflate the first balloon 103. The first pump 105 may either be electronically or mechanically connected to a controller 701 configured drive the first pump 105. In some embodiments, a user selects inputs a desired inflation pressure for the first balloon 103 into the controller 701 which drives the first pump 105 inflating the first balloon 103.

The second balloon catheter 111 may be mechanically connected to a second pump 115 configured to inflate the second balloon 113. The second pump 115 may either be electronically or mechanically connected to the controller 701 configured drive the second pump 115. The second balloon catheter 111 may either be electronically or mechanically connected to a monitor 702 (e.g., pressure measuring device) configured to measure the subclavian pressure. In some embodiments, a user selects inputs a desired inflation pressure for the second balloon 113 into the controller 701 which drives the second pump 115 inflating the second balloon 113. The monitor 702 may continuously or discretely measure the pressure at an area near the second balloon 113 (e.g., the subclavian pressure). In some embodiments, a user may cause the second balloon 113 to be deflated using the controller 701 in response to an increase in the subclavian pressure. In some embodiments, the monitor 702 may either be electronically or mechanically connected to the controller 701. In some embodiments, the monitor 702 is configured to transmit the subclavian pressure to the controller 701. In some embodiments, the controller 701 is configured to automatically cause the second balloon 113 to be deflated in response to an increase in the subclavian pressure.

The third balloon catheter 501 may be mechanically connected to a third pump 505 configured to inflate the third balloon 503. The third pump 505 may either be electronically or mechanically connected to the controller 701 configured drive the third pump 505. The third balloon catheter 501 may either be electronically or mechanically connected to a monitor 702 (e.g., pressure measuring device) configured to measure the pulmonary artery (“PA”) and femoral vein pressures. In some embodiments, a user selects inputs a desired inflation pressure for the third balloon 503 into the controller 701 which drives the third pump 505 inflating the third balloon 503. The monitor 702 may continuously or discretely measure the pressure at an area near the third balloon 503 (e.g., the pulmonary artery pressure). The monitor 702 may continuously or discretely measure the pressure at an area near the inlet 301 of the first catheter 101 (e.g., the femoral vein pressure). In some embodiments, a user may cause the third balloon 503 to be deflated using the controller 701 in response to an increase in with the pulmonary artery pressure or femoral vein pressure. In some embodiments, the monitor 702 may either be electronically or mechanically connected to the controller 701. In some embodiments, the monitor 702 is configured to transmit the pulmonary artery and femoral vein pressures to the controller 701. In some embodiments, the controller 701 is configured to automatically cause the third balloon 503 to be deflated in response to an increase in either the pulmonary artery or femoral vein pressures.

### Catheter-Based Methods of Treatment

As described herein, the present disclosure provides methods for the treatment of heart failure, particularly acute decompensated heart failure (ADHF), using a balloon catheter system. With the guidance of hemodynamic parameter indicators, intermittent occlusion of veins by balloon inflation may be performed so as to reduce the amount of blood returning to the heart, which may reduce the preload of the heart to relieve one or more symptoms of heart failure. For example, studies conducted by the inventor(s) have shown that fluid retention in patients can be improved by use of the catheter systems as described and prescribed herein.

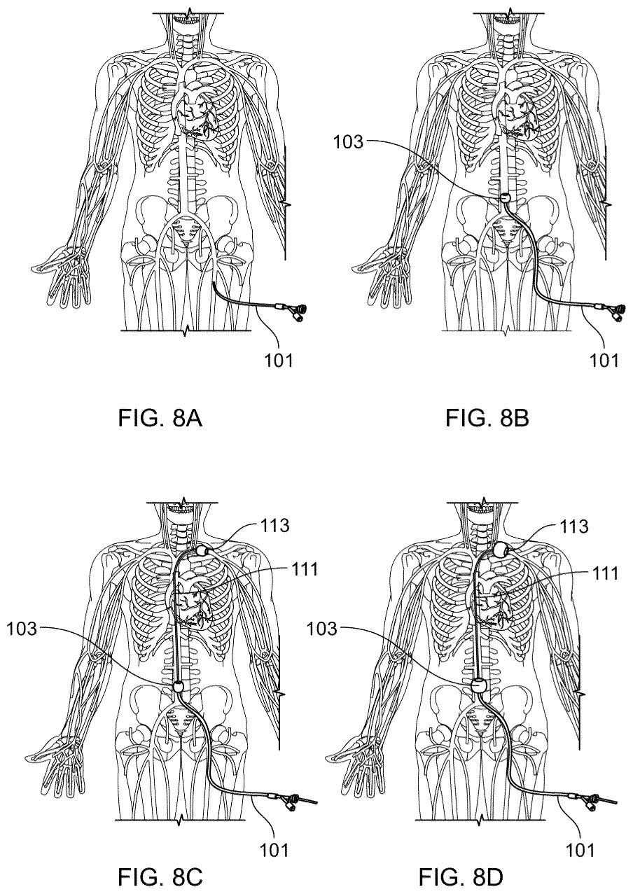

FIGS. 8A-8D are illustrations depicting a method for using the balloon catheter systems described herein to treat heart failure, according to some embodiments. FIG. 8A shows the step of introducing the balloon catheter system 100 into the femoral vein through a puncture site. FIG. 8B shows the step of advancing the first balloon 103 of the first balloon catheter 101 to the first target location at the inferior vena cava (“IVC”) proximal to either the left or right renal veins. FIG. 8C shows the step of advancing the second balloon 113 of the second balloon catheter 111 to the second target location proximal to either the left or right subclavian vein. FIG. 8D shows the step of inflating the first balloon 103 and second balloon 113 of the balloon catheter system 100.

In some embodiments, the method further comprises enlarging the puncture site using the detachable sheath 601 and advancing the first balloon 103 of the first balloon catheter 101 through the detachable sheath 601 to the first target location. After the first balloon 103 is positioned at the first target location the detachable sheath 601 may be removed, such as, for example, by tearing the detachable sheath 601 using the first detachable arm 602 and a second detachable arm 603.

In some embodiments, the first balloon 103 is advanced to the first target location using a guidewire. In some embodiments, the second balloon 113 is advanced to the second target location using a guidewire (“GW”).

In some embodiments, the first balloon 103 comprises further at least one radio-opaque marker 311 allowing a user to visualize the first balloon 103 under radiography or fluoroscopy, such that the user may accurately guide the first balloon 103 to the first target location. In some embodiments, the second balloon 113 comprises further at least one radio-opaque marker 401 allowing a user to visualize the second balloon 113 under radiography or fluoroscopy, such that the user may accurately guide the second balloon 113 to the second target location.

In some embodiments, intermittent occlusion of one or more veins reduces venous blood backflow to the heart and/or reduces the pumping burden for the heart. In some embodiments, inflation of the first balloon 103 creates a low-pressure area at or below the renal vein(s), improving blood and lymphatic return and organ function. For example, blood pressure at or below the renal vein(s), as measured by the first balloon catheter 101, may be reduced by about 5%, 10%, 15%, 20%, 25%, 30%, 35%, 40%, 45%, 50%, 55%, 60%, 65%, 70%, 75%, 80%, 85%, 90%, 95%, or any range therebetween with the first balloon 103 inflated. In some embodiments, the low-pressure area in the renal vein promotes renal circulation, speeds up urination, and/or reduced fluid retention in the body. In some embodiments, inflation of the first balloon 103 stimulates the vagus nerve causing enhanced blood accommodation in the lower extremities and abdomen. In some embodiments, stimulation of the vagus nerve can reduce venous blood backflow to the heart and reduce the pumping burden for the heart.

In some embodiments, the first balloon 103 of the first balloon catheter 101 is positioned at a first target location in the inferior vena cava (“IVC”) proximal to either the left or right renal veins. In some embodiments, the first balloon 103 is positioned away from the left or right renal vein by at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or any values therebetween. In some embodiments, the first balloon 103 is positioned away from the left or right renal vein by at most about 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some embodiments, the first balloon 103 may be from about 1 mm to about 25 mm away from the left or right renal vein. In some embodiments, the first balloon 103 may be from about 5 mm to about 25 mm away from the left or right renal vein. In some embodiments, the first balloon 103 may be from about 10 mm to about 25 mm away from the left or right renal vein. In some embodiments, the first balloon 103 may be from about 15 mm to about 25 mm away from the left or right renal vein. In some embodiments, the first balloon 103 may be from about 15 mm to about 20 mm away from the left or right renal vein.

In some embodiments, the first balloon 103 may be inflated for a period of at least about 1 second (“sec”), 2 sec, 3 sec, 4 sec, 5 sec, 6 sec, 7 sec, 8 sec, 9 sec, 10 sec, 20 sec, 30 sec, 40 sec, 50 sec, 60 sec, 70 sec, 80 sec, 80 sec, 100 sec, or any values therebetween. In some embodiments, the first balloon 103 may be inflated for a period of at most about 100 sec, 90 sec, 80 sec, 70 sec, 60 sec, 50 sec, 40 sec, 30 sec, 20 sec, 10 sec, 9 sec, 8 sec, 7 sec, 6 sec, 5 sec, 4 sec, 3 sec, 2 sec, 1 sec, or any values therebetween. In some embodiments, the first balloon 103 may be inflated for a period from about 1 sec to about 60 sec. In some embodiments, the first balloon 103 may be inflated for a period from about 10 sec to about 40 sec. In some embodiments, the first balloon 103 may be inflated for a period from about 20 sec to about 40 sec. Inflation of the first balloon 103 may occlude the inferior vena cava near the renal veins by about 10%, 20%, 30%, 40%, 50%, 60%, 70%, 80%, 90%, or any range therebetween.

In some embodiments, intermittent occlusion of one or more veins reduces venous blood backflow to the heart and/or reduces the pumping burden for the heart. In some embodiments, inflation of the second balloon 113 creates a low-pressure area near the left internal jugular vein and thoracic duct, improving return to the SVC and the right atrium of the heart, increases lympathic reflow, and/or reduces fluid retention in the body. For example, blood pressure near the left internal jugular vein and thoracic duct, as measured by the second balloon catheter 111, may be reduced by about 5%, 10%, 15%, 20%, 25%, 30%, 35%, 40%, 45%, 50%, 55%, 60%, 65%, 70%, 75%, 80%, 85%, 90%, 95%, or any range therebetween with the second balloon 113 inflated. In some embodiments, inflation of the second balloon stimulates the vagus nerve, causing improved cardiac remodeling and function. Stimulation of the vagus nerve can cause vasodilation which may increase blood accommodation in the venous bed and reduces heart preload. In some embodiments, stimulation of the vagus nerve can reduce venous blood backflow to the heart and reduce the pumping burden for the heart.

In some embodiments, the second balloon 113 may be inflated for a period of at least about 1 second (“sec”), 2 sec, 3 sec, 4 sec, 5 sec, 6 sec, 7 sec, 8 sec, 9 sec, 10 sec, 20 sec, 30 sec, 40 sec, 50 sec, 60 sec, 70 sec, 80 sec, 80 sec, 100 sec, or any values therebetween. In some embodiments, the second balloon 113 may be inflated for a period of at most about 100 sec, 90 sec, 80 sec, 70 sec, 60 sec, 50 sec, 40 sec, 30 sec, 20 sec, 10 sec, 9 sec, 8 sec, 7 sec, 6 sec, 5 sec, 4 sec, 3 sec, 2 sec, 1 sec, or any values therebetween. In some embodiments, the second balloon 113 may be inflated for a period from about 1 sec to about 60 sec. In some embodiments, the second balloon 113 may be inflated for a period from about 10 sec to about 40 sec. In some embodiments, the first balloon 113 may be inflated for a period from about 10 sec to about 30 sec. Inflation of the second balloon 113 may occlude the subclavian vein by about 10%, 20%, 30%, 40%, 50%, 60%, 70%, 80%, 90%, or any range therebetween.

In some embodiments, the method further comprises the step of measuring the pressure of at the subclavian vein using the second balloon catheter 111. The measured pressure may be used to determine whether to inflate or deflate the first balloon 103 and/or second balloon 113. Inflation or deflation of the first balloon 103 may cause a pressure change (e.g., modulates the pressure) at or below the renal vein. For example, inflation of the first balloon 103 may cause an increase in pressure below the renal vein. In some embodiments, when the pressure at the renal vein increases by about 1 mmHg, 2 mmHg, 3 mmHg, 4 mmHg, 5 mmHg, 6 mmHg, 7 mmHg, 8 mmHg, 9 mmHg, 10 mmHg, or any values therebetween, the first balloon 103 is deflated. In some embodiments, when the pressure at the renal vein increases by about 1 mmHg to about 10 mmHg, the first balloon 103 is deflated. In some embodiments, when the pressure at the renal vein increases by about 3 mmHg to about 5 mmHg, the first balloon 103 is deflated.

Inflation or deflation of the second balloon 113 may cause a pressure change (e.g., modulates the pressure) near the left internal jugular vein and thoracic duct. For example, inflation of the second balloon 113 may cause an increase in pressure near the left internal jugular vein and thoracic duct. In some embodiments, when the pressure at the left internal jugular vein and thoracic duct increases by about 1 mmHg, 2 mmHg, 3 mmHg, 4 mmHg, 5 mmHg, 6 mmHg, 7 mmHg, 8 mmHg, 9 mmHg, 10 mmHg, or any values therebetween, the second balloon 113 is deflated. In some embodiments, when the pressure at the left internal jugular vein and thoracic duct increases by about 1 mmHg to about 10 mmHg, the second balloon 113 is deflated. In some embodiments, when the pressure at the left internal jugular vein and thoracic duct increases by about 5 mmHg to about 10 mmHg, the second balloon 113 is deflated.

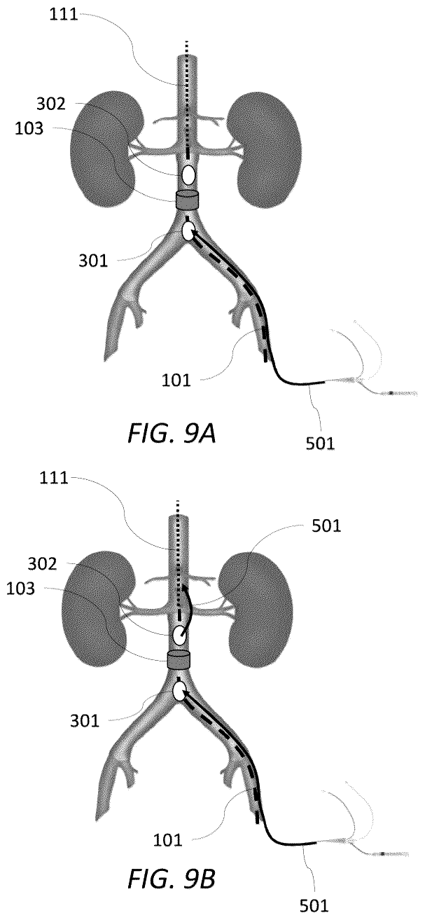

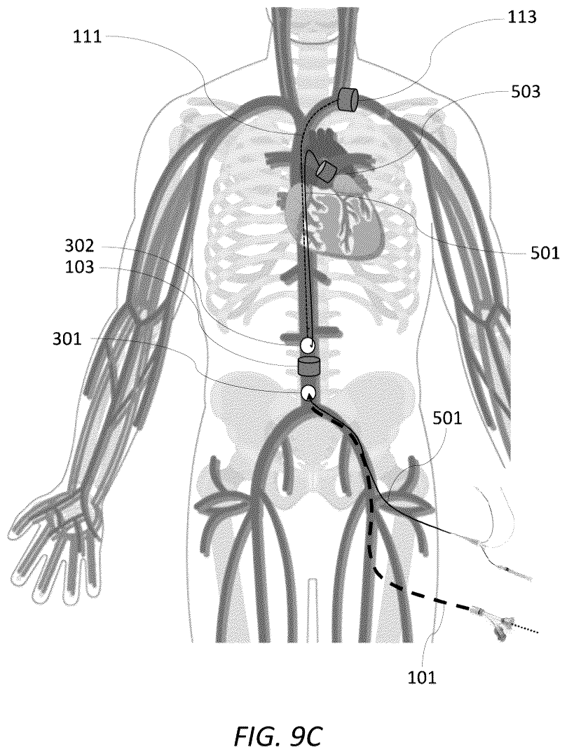

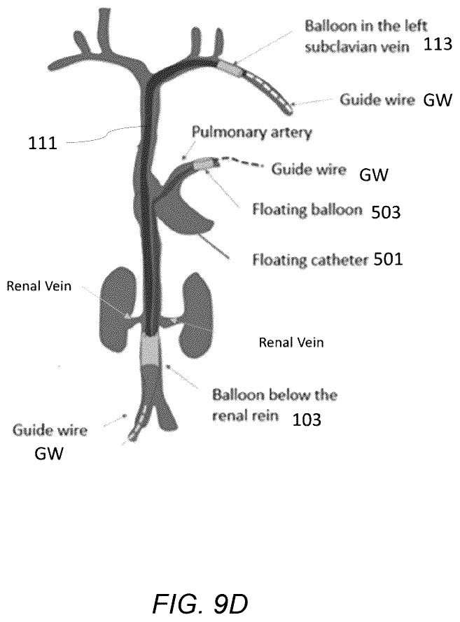

FIGS. 9A-9D are illustrations depicting a method for advancing a pulmonary artery catheter to the third target location at the pulmonary artery. FIG. 9A shows the step of introducing the third balloon catheter 501 into the inlet 301 of the first balloon catheter 101. The third balloon catheter 501 is advanced through the third lumen 203 of the first balloon catheter 101 until exiting through the outlet 302 of the first balloon catheter 101, as shown in FIG. 9B. FIG. 9C shows the step of advancing the third balloon 503 of the third balloon catheter 501 to the third target location proximal to the pulmonary artery. Once positioned at the third target location, the third balloon 503 is inflated and the pressure at one or more of the pulmonary artery or right atria may be measured. FIG. 9D shows the final positioning of the balloon catheter system for use to treat heart failure.

In some embodiments, the third balloon 503 is advanced to the third target location using a guidewire.

In some embodiments, the outlet 302 of the first balloon catheter 101 is positioned in the inferior vena cava below the left or right renal veins. In some embodiments, the outlet 302 is positioned below the left or right renal vein by at least about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or any values therebetween. In some embodiments, the outlet 302 is positioned below the left or right renal vein by at most about 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm, or any values therebetween. In some embodiments, the outlet 302 may be from about 1 mm to about 25 mm below the left or right renal vein. In some embodiments, the outlet 302 may be from about 5 mm to about 20 mm below the left or right renal vein. In some embodiments, the outlet 302 may be from about 5 mm to about 15 mm below the left or right renal vein. In some embodiments, the outlet 302 is about 10 mm below the left or right renal vein.

In some embodiments, the third balloon 503 comprises further at least one radio-opaque marker allowing a user to visualize the third balloon 503 under radiography or fluoroscopy, such that the user may accurately guide the third balloon 503 to the second target location.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing the present disclosure. It is therefore contemplated that the present disclosure shall also cover any such alternatives, modifications, variations or equivalents. It is intended that the following claims define the scope of the present disclosure and that methods and structures within the scope of these claims and their equivalents be covered thereby.

1.-105. (canceled)

106. A method for treatment of a cardiovascular disease of a subject, the method comprising using a catheter system comprising:
a first catheter comprising;
a first balloon,
a first lumen, configured to inflate the first balloon,
a second lumen, and
a third lumen,
a second catheter comprising a second balloon and an inflation lumen configured to inflate the second balloon, wherein the second lumen of the first catheter is configured to accept the second catheter for advancement and retraction therethrough, and
wherein the third lumen of the first catheter is configured to accept a third catheter for advancement and retraction therethrough, wherein the third catheter comprises a third balloon and an inflation lumen configured to inflate the third balloon, wherein the third balloon is configured to be positioned at or near a pulmonary artery of the subject.

107. The method of claim 106, wherein the method comprises inserting the catheter system into a femoral vein of the subject;
positioning the tip of the first catheter of the catheter system at or near a renal vein of the subject;
positioning the second balloon of the second catheter of the catheter system at or near a subclavian vein of the subject; and
inflating the first balloon and the second balloon;
wherein inflation of the second balloon creates a low-pressure area at the left internal jugular vein and thoracic duct of the subject, and
wherein inflation of the first balloon creates a low-pressure area at the renal vein of the subject.

108. The method of claim 106, wherein the method comprises:
advancing the first catheter into a femoral vein of the subject;
positioning the first balloon at an inferior vena cava (IVC) of at or near a renal vein of the subject;
advancing the second catheter through the first catheter;
positioning the second balloon of the second catheter at a subclavian vein of the subject; and
inflating the second balloon, thereby creating a low-pressure area at one or more of the left internal jugular vein or the thoracic duct of the subject.

109. The method of claim 106, wherein inflation of the second balloon stimulates a vagus nerve of the subject.

110. The method of claim 106, wherein inflation of the first balloon stimulates a vagus nerve of the subject.

111. The method of claim 106, wherein inflating the first balloon comprises maintaining inflation of the first balloon for 20-40 seconds before deflating the first balloon.

112. The method of claim 106, wherein inflating the second balloon comprises maintaining inflation of the second balloon for 10-30 seconds before deflating the second balloon.

113. The method of claim 106, wherein positioning the tip of the first catheter at or near the renal vein comprises advancing the first catheter through vasculature of the subject over a guidewire.

114. The method of claim 106, wherein positioning the second balloon at or near the subclavian vein of the subject comprises advancing the second catheter through vasculature of the subject over a guidewire.

115. The method of claim 106, further comprising:
inserting the third catheter comprising the third balloon into the third lumen of the catheter system;
positioning the third balloon of the third catheter at or near the pulmonary artery;
inflating the third balloon; and
measuring a pulmonary artery pressure of the subject with the catheter system.

116. The method of claim 115, wherein positioning the third balloon of the third catheter at or near the pulmonary artery comprises advancing the third catheter through vasculature of the subject over a guidewire.

117. The method of claim 116, further comprising monitoring at least one hemodynamic parameter of the subject with the third catheter.

118. The method of claim 117, wherein the at least one hemodynamic parameter is one or more of heart rate, blood pressure, stroke volume, cardiac output, or total peripheral resistance.

119. The method of claim 106, further comprising monitoring pressure at a right atria of the subject with the catheter system.

120. The method of claim 106, further comprising monitoring pressure at a pulmonary artery of the subject with the catheter system.

121. The method of claim 106, further comprising monitoring pressure at one or more of an inferior vena cava or femoral vein with the catheter system.

122. The method of claim 106, wherein the cardiovascular disease is heart failure or acute heart failure.

123. The method of claim 109, wherein stimulating the vagus nerve of the subject causes one or more of a subclavian vein, a left internal jugular vein or thoracic duct, an inferior vena cava, or a renal vein to expand.

124. The method of claim 109, wherein stimulating the vagus nerve of the subject reduces venous blood backflow to the heart of the subject, reduces heart pumping burden of the subject, or both.

125. A catheter system for treatment of a cardiovascular disease of a subject, the catheter system comprising:
a first catheter comprising;
a first balloon,
a first lumen, configured to inflate the first balloon,
a second lumen, and
a third lumen,
a second catheter comprising a second balloon and an inflation lumen configured to inflate the second balloon, wherein the second lumen of the first catheter is configured to accept the second catheter for advancement and retraction therethrough, and
wherein the third lumen of the first catheter is configured to accept a third catheter for advancement and retraction therethrough, wherein the third catheter comprises a third balloon and an inflation lumen configured to inflate the third balloon.
